(19) United States
(12) Patent Application Publication
(10) Pub. No.: US 2026/0166377 A1
(43) Pub. Date: Jun. 18, 2026

(54) METHOD AND APPARATUS FOR A HEALTH MONITORING SYSTEM

(71) Applicant: Aspire Innovations Corp., Phoenix, AZ (US)

(72) Inventors: Reymundo Perez, Phoenix, AZ (US); Coby Hochstein, Chandler, AZ (US)

(73) Assignee: Aspire Innovations Corp., Phoenix, AZ (US)

(21) Appl. No.: 19/411,323

(22) Filed: Dec. 7, 2025

Related U.S. Application Data
(60) Provisional application No. 63/729,447, filed on Dec. 8, 2024.

(51) Int. Cl. A63B 24/00; A61B 5/00

(52) U.S. Cl. CPC A63B 24/0062; A61B 5/0002; A61B 5/7285; A61B 2503/10; A61B 2560/0209; A61B 2560/0475; A61B 2562/0219; A63B 2220/53

(57) ABSTRACT

A health monitoring system includes a plurality of athletes participating in a competitive activity. A plurality of sensors is indirectly coupled to each of the plurality of athletes respectively and each sensor detects a physical aspect of each athlete. The health monitoring system further includes a sideline controller that is wirelessly coupled to one of the plurality of sensors, where the one of the plurality of sensors receives physical aspect data from the remaining sensors and transmits all physical aspect data collected from each of the plurality of athletes to the sideline controller. A storage device such as an encrypted storage cloud is wirelessly coupled to the sideline controller to receive the physical aspect data collected from each of the plurality of athletes. The sideline controller is predictive of potential harm brought to one or more of the plurality of athletes via analysis of the collected physical aspect data.

### BACKGROUND OF THE INVENTION

Student athletic competitions are among the most popular of the several types of competitions both from the competitor and spectator points of view. Sadly, however, contact and non-contact sports alike have contributed and continue to contribute to debilitating physical (e.g., brain) injuries for those athletes who participate in them. As reported by the National Center for Catastrophic Sport Injury Research (NCCSIR), football-related activities were responsible for 52% of the total number of sport-related catastrophic injuries reported among student athletes for the 2022-2023 academic year. 83% of the total injuries reported by NCCSIR occurred during high school football events while college football events contributed 17% of the total injuries reported. 74% of all traumatic injuries resulting in permanent disability or death for the 2022-2023 academic year occurred during football-related activities according to the NCCSIR. According to the U.S. Centers for Disease Control and Prevention (CDC), youth sports with the highest rate of concussions occur in tackle football and wrestling for boys and in basketball for girls with virtually no conventional means for the evaluation of the cumulative effects of such collisions over time.

While the National Collegiate Athletics Association (NCAA) continues to improve its health evaluation protocols and the CDC continues its concussion and other brain injury awareness campaigns, concussion evaluation protocols implemented by the NCAA are currently based on single-impact events and do not consider nor are they capable of considering the cumulative effects of single-impact events occurring over time. Furthermore, concussion protocols are implemented subjectively by medical personnel who may inject a significant amount of error into the evaluation process due to the lack of quantitative measurements currently available.

Limitations associated with conventional impact measurement devices may include inadequate acceleration detection ranges between about +/−32 times the standard acceleration due to gravity near the Earth's surface (+/−32 g) when sports-related impacts exceeding 100 g and helmet-to-helmet impacts exceeding 150 g are typical. In such instances, conventional impact measurement devices may clip or saturate during any impact events that exceed their dynamic range thereby failing to capture critical impact measurement data.

Conventional impact measurement devices may additionally exhibit poor battery life due to the continuous high-speed sampling (e.g., at or above one thousand samples per second) that may be required to capture short-duration impact events. Such high-speed, continuous sampling may deplete battery-powered impact measurement devices in hours, not days, which may render them impractical for game-day use. Continuous sampling may also lead to false positive impact events generated during normal running and jumping that may generate excessive data storage and alert fatigue that may then be ignored by trainers and coaching staff.

Conventional impact measurement devices may further require physical interfacing (e.g., via a universal serial bus (USB)), which may require their removal in order to download pertinent data. As such, real-time monitoring may be impossible which may lead to intolerable delays before dangerous impact and other measurement data may be made known to coaching and training staff members.

Conventional impact measurement devices may not combine other measurement capabilities such as hydration deprivation, pulse, blood pressure, blook oxygen and body temperature to name only a few. As such, conventional impact measurement devices may not combine impact data with other measured data to facilitate a holistic approach to determine the health and well-being of connected athletes in real time.

Efforts continue, therefore, to develop health monitoring systems and related detection mechanisms that facilitate objectivity while determining not only single-event impact and other monitored data on the health of an athlete, but that also may track and predict the cumulative adverse effects of such impacts and other monitored data over time.

### SUMMARY OF THE INVENTION

To overcome limitations in the prior art, and to overcome other limitations that will become apparent upon reading and understanding the present specification, various embodiments of the present invention disclose methods and apparatus for monitoring the health of a plurality of athletes participating in a competitive activity. Each athlete is placed into proximity of a sensor that may be configured to measure a plurality of physical aspects including head impact, hydration, pulse, blood pressure, blood oxygenation, body temperature, weight/body mass index (BMI) and foot (or other body part) impact data related to each athlete. Each sensor is configured to transmit its respective data to a sideline controller thus allowing coaches and trainers to monitor the data in an effort to detect potential harm and injury that may be brought to each athlete by virtue of his/her participation in the competition. All sensor data may be transmitted to an encrypted storage cloud thereby providing access to all sensor data to anyone that has been allowed such access to the cloud. As such, for example, prior medical history relating to each athlete may be combined with the sensor data to further enhance the injury-predicting capability of the health monitoring system.

In accordance with one embodiment of the invention, a health monitoring system comprises a plurality of athletes participating in a competitive activity. The health monitoring system further includes a plurality of sensors indirectly coupled to each of the plurality of athletes respectively, where each sensor detects a physical aspect of each athlete. The health monitoring system further includes a sideline controller wirelessly coupled to one of the plurality of sensors, where the one of the plurality of sensors receives physical aspect data from the remaining sensors and transmits all physical aspect data collected from each of the plurality of athletes to the sideline controller. The health monitoring system further includes a storage device coupled to the sideline controller and is coupled to receive the physical aspect data collected from each of the plurality of athletes. The sideline controller may predict potential harm brought to one or more of the plurality of athletes via analysis of the collected physical aspect data.

In accordance with another embodiment of the invention, a method of monitoring the health of a plurality of athletes comprises initializing a plurality of operational modes associated with an inertial measurement unit (IMU) coupled in proximity to each of the plurality of athletes, each operational mode being indicative of a level of acceleration measured by the IMU, modifying an overhead of a microcontroller in accordance with each operational mode, modifying power consumed by the IMU in accordance with each operational mode and recording motion detected by the IMU when the measured level of acceleration exceeds a configurable threshold.

### BRIEF DESCRIPTION OF DRAWINGS

Various aspects and advantages of the invention will become apparent upon review of the following detailed description and upon reference to the drawings in which:

FIG. 1 illustrates a block diagram of a health monitoring system in accordance with an embodiment of the invention;

FIG. 2 illustrates a block diagram of a sensor included within the health monitoring system of FIG. 1 in accordance with an embodiment of the invention; and

FIG. 3 illustrates a state diagram of operation of the sensor of FIG. 2 in accordance with an embodiment of the invention.

### DETAILED DESCRIPTION OF THE INVENTION

A portion of the disclosure of this patent document contains material which is subject to copyright protection. The copyright owner has no objection to the facsimile reproduction by anyone of the patent document or the patent disclosure, as it appears in the U.S. Patent & Trademark Office patent file or records, but otherwise reserves all copyright rights whatsoever.

Generally, the various embodiments of the present invention are applied to systems for concurrently monitoring the health of athletes during their participation in athletic competitions. These health monitoring systems may be comprised of one or more devices, each being capable of collecting a wide variety of physiological data, which may then be analyzed in real time to issue alerts when harmful, single-event thresholds have been exceeded. Further, single-event data may be tracked over time to determine whether cumulative harmful thresholds may have been exceeded.

The one or more health monitoring devices may be included within a personal area network (PAN) so as to provide health monitoring data that may be related to a team of athletes (e.g., football players) that are capable of inter-network communication (e.g., via Bluetooth low energy (BLE) or a thread-based mesh network). In one embodiment, one such device may operate as a master device with all other devices slaved to the master device. In such an instance, the master device may upload its data along with the other data collected from the slave devices via a wireless network (e.g., via BLE, WiFi, thread-based mesh) to an upload device (e.g., a phone, tablet, desktop or laptop) when the upload device is within range of the master device. Alternatively, each health monitoring device may independently upload its respective data to the upload device instead.

In one embodiment, the health monitoring slave devices may not include a user interface but may only be included within the network as data collection devices that may be polled as needed by the health monitoring master device. Each of the health monitoring slave and master devices may be paired upon distribution to the individual athletes that form a team of athletes who are to be monitored in real time during the team competition.

An upload device (e.g., a sideline controller implemented as a phone, tablet, desktop or laptop) may retrieve all data from the master device (e.g., which may include data uploaded to the master device from all slave devices) once the master device is within an upload range of the sideline controller. Alternately, the sideline controller may retrieve data from each slave device individually. The sideline controller may include a user interface (e.g., a messaging interface implemented by a smartphone) that may be accessible by the coaching staff when the coaching staff needs to query monitored data whether from a single athlete or a group of two or more athletes. The collected data may be aggregated for the entire team and may be highlighted to alert the coaching staff as to any athlete who may be approaching any single event, or multiple event, danger threshold. In one embodiment, the sideline controller may be configured to register an audible, visual and/or tactile alert autonomously once any one or more athletes may be approaching and/or exceeding danger thresholds, where such danger thresholds may be predetermined within the sideline controller. In such an instance, the coaching staff may be apprised of the endangered athlete(s) so that an immediate intervention may be scheduled before any further harm may be imposed upon the one or more athletes.

The sideline controller may store the aggregated data along with any associated alerts within internal memory until such time that the aggregated data and associated alerts may be uploaded to a central storage system (e.g., a cloud-based storage system). Such an upload may occur via a wireless (e.g., via WiFi or Cellular) network or a wired (e.g., Ethernet) network. The central storage system may receive uploaded data from any one or more associated sideline controllers and may be fault tolerant so as to preserve all data in the event of a cloud failure. The central storage system may allow access to athlete data who may be approaching endangerment thresholds and may further provide autonomous notification alerts of such endangered athletes to interested persons (e.g., coaches, trainers, medical personnel and/or family members). The central storage system may support administrative settings that may be selected via a user interface. Such administrative settings may allow a selected administrator to provide access privileges to selected users who then may be allowed to customize single-event and/or multiple-event thresholds, notification settings and other applicable settings.

Access privileges may be granted, for example, to officials who may be associated with the National Collegiate Athletic Association (NCAA), the National Football League (NFL), the Centers for Disease Control and Prevention (CDC) and others who may be interested in improving assessment protocols (e.g., concussion protocols) through the use of data stored within the central storage system. Coaches and trainers may be granted access to the central system to allow the review of data and associated alarms associated with their respective team members. Furthermore, coaches and trainers may be allowed to review other teams'data so that data sets gathered in relation to their home team athletes may be compared and contrasted with data associated with the other teams'athletes so as to better identify average trends and/or outliers. As such, a “league view” may accommodate viewing of data associated with an individual team, whereas a “team view” may accommodate viewing of data associated with an individual athlete. Likewise, family members may be granted access to the central storage system and may be given the ability to forward family-member data to medical practitioners who may then provide a critical diagnosis that may be made more accurate through the use of such data when compared and contrasted with the medical history.

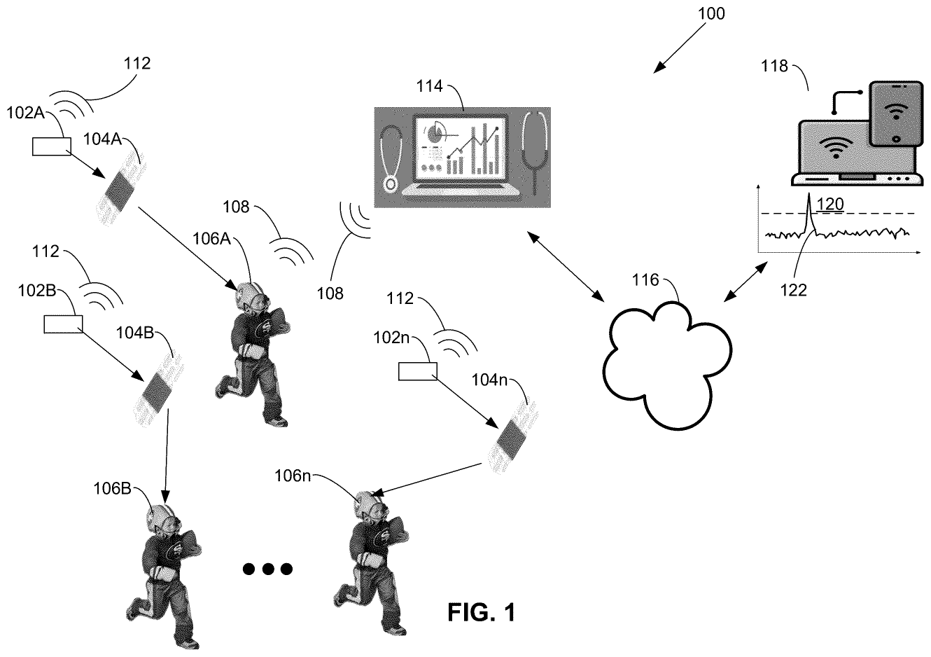

Turning to FIG. 1, health monitoring system 100 is exemplified which may include one or more sensors 102A and 102B-102n that may be incorporated into associated applicators (e.g., adhesive-style applicators 104A, and 104B-104n, respectively) and configured to measure a physical aspect (e.g., position, orientation, velocity and acceleration) of a particular body part (e.g., head or foot) of its respective athlete. Sensors 102A and 102B-102n may further include the capability to measure other physiological attributes of each athlete such as hydration, pulse, blood pressure, blood oxygen level and body temperature and to provide data indicative of such measured physiological attributes. In one embodiment, applicators 104A and 104B-104n may not attach directly to a body part of respective athletes 106A and 106B-106n, but may rather indirectly attach (e.g., via an adhesive-based or non-adhesive-based attachment point within an interior of each helmet of each athlete 106A and 106B-106n, respectively). As such, sensors 102A and 102B-102n may be more apt to remain within proximity to athletes 106A and 106B-106n, respectively, thereby increasing the integrity of data collected by sensors 102A and 102B-102n.

As discussed above, sensor 102A may be identified and provisioned within health monitoring system 100 as a master sensor. Sensors 102B-102n may, on the other hand, be identified and provisioned as slave sensors. As such, sensor data may be collected by slave sensors 102B-102n during competition and transmitted via a wireless network (e.g., a Bluetooth low-energy (BLE) or thread-based mesh network 112) to master sensor 102A once network 112 is available to relay such slave sensor data. Master sensor 102A may then transmit all sensor data to a sideline controller (e.g., sideline controller 114) for real-time analysis. Alternatively, each sensor 102A and 102B-102n may each operate as master sensors thereby directly transmitting their respective data to sideline controller 114 instead.

A wireless network (e.g., a BLE, WiFi or thread-based mesh network 108) may be used to transmit all sensor data (e.g., master sensor 102A data and slave sensor 102B-102n data) to sideline controller 114 (e.g., smart phone, tablet, or desktop). In one embodiment, wireless network 112 and 108 may be the same network. Sideline controller 114 may be used by coaching/training personnel to monitor athletes for single-event or multiple-event injuries that may exceed a safe threshold. In one embodiment, data gathered by sensors 102A and 102B-102n may be analyzed via sideline controller 114 to determine whether a threat of concussion or another physical malady exists for any athlete 106A and 106B-106n, respectively. If a threat is detected, sideline controller 114 may alarm a coach/trainer as to a recommended plan of action.

Data collected by sideline controller 114 may be uploaded to a storage cloud (e.g., encrypted storage cloud 116) which may then be made available to be accessed by any device 118 that may be authenticated to storage cloud 116. In one embodiment, device 118 may include medical history information that may be exclusive to one or more athletes 106A and 106B-106n. As such, prior medical history of a particular athlete may be paired with sensor data associated with that particular athlete to determine whether data gathered by sensors 102A and 102B-102n can be shown to be problematic (e.g., event 122 may exceed danger threshold 120). In such an instance, an alarm may be generated by device 118 and transmitted to sideline controller 114 (e.g., via encrypted storage cloud 116) for further action to be taken by coaches/trainers associated with athletes 106A and 106B-106n.

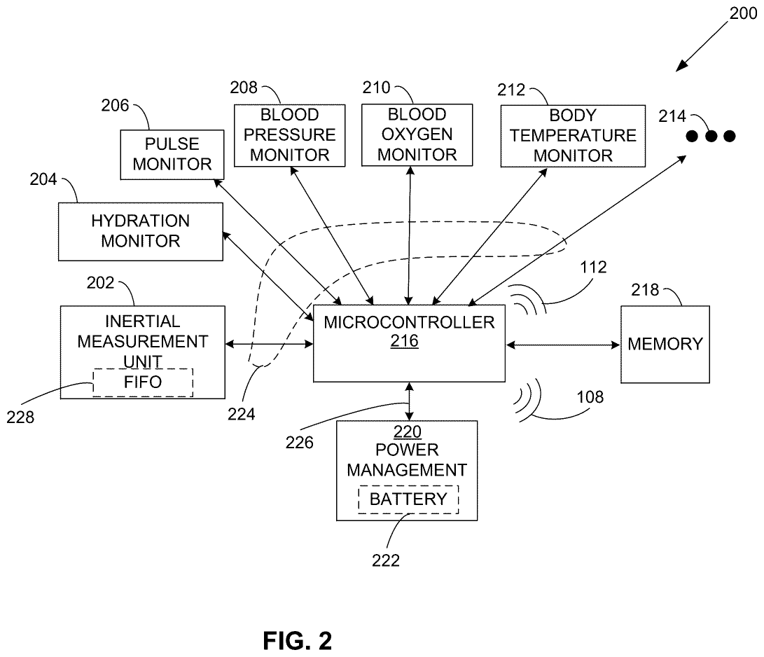

Turning to FIG. 2, sensor 200 (e.g., as discussed above in relation to sensors 102A and 102B-102n of FIG. 1) is exemplified as including memory (e.g., flash memory 218), power management 220 having battery 222, microcontroller 216 and one or more monitors such as the ability to measure/monitor position, orientation, velocity and acceleration (e.g., via inertial measurement unit (IMU) 202) of an athlete's (e.g., athletes 106A and 106B-106n of FIG. 1) head, foot or other body part. Sensor 200 may also include one or more other monitors, such as the ability to measure/monitor: 1) the hydration level of an athlete (e.g., via hydration monitor 204); 2) the pulse rate of an athlete (e.g., via pulse monitor 206); 3) the blood pressure of an athlete (e.g., via blood pressure monitor 208); 4) the blood oxygen level of an athlete (e.g., via blood oxygen monitor 210); 5) the body temperature of an athlete (e.g., via body temperature monitor 212); and/or 6) monitors 214 (e.g., weight and body mass monitors) to name only a few types of monitors that may be included within sensor 200.

Each one or more interfaces 224 may, for example, include a serial peripheral interface (SPI) that may be used for high-speed, full duplex, synchronous communications between each one or more monitors 202-214 and microcontroller 216. Further, each of monitors 202-214 may be connected to an interrupt and control interface (e.g., interface 224) which as discussed in more detail below may relieve microcontroller 216 of the requirement to continuously poll for monitored data. Instead, each of monitors 202-214 may be initialized to operate autonomously with respect to microcontroller 216 and may interrupt microcontroller 216 (e.g., using multi-level priority interrupts via interface 224) when monitored data is available.

IMU 202 may provide several modes of operation that may maximize performance when used as a tool to support health evaluation of athletes via continuous monitoring/measurement of position, orientation, velocity and/or acceleration data. In addition, IMU 202 may be initialized by microcontroller 216 to select its mode of operation autonomously with respect to microcontroller 216 by virtue of the automatic detection of position, orientation, velocity and/or acceleration changes experienced by the athlete (or the athlete's equipment) to which IMU 202 may be attached. For example, IMU 202 may operate in an impact capture mode of operation capable of measuring impact events to the athlete's helmet that may be caused by accelerations typically experienced in sports-related activities such as football, hockey, boxing, Lacrosse, etc. As such, for example, the dynamic range of IMU 202 during the impact capture mode of operation may be such that impact data well in excess of 200 g may be detected and sampled at a high rate (e.g., at a sampling rate of about 3200 Hz) and recorded without the possibility of clipping or saturating such as may be experienced by conventional impact detection devices.

During an absence of impact events (e.g., no impact events detected within 20-500 milli-seconds of last detected impact event), a reduction in power consumption may be realized by IMU 202 autonomously placing itself into an active mode of operation whereby sustained motion resulting in nominal acceleration forces (e.g., at or below about 30 g) may nevertheless be monitored using a reduced sampling rate (e.g., about 200 Hz). In order to further reduce power consumption in an absence of sustained motion (e.g., no sustained motion detected within 30 seconds of last sustained motion detection), IMU 202 may autonomously place itself into a sleep mode of operation whereby minimal acceleration forces (e.g., at or below about 0.5 g) may be monitored using a reduced sampling rate (e.g., about 12.5 Hz). Generally speaking, by autonomously monitoring an athlete's position, orientation, velocity and/or acceleration via IMU 202, sensor 200 may be transitioned into decreasing power consumption modes so that battery 222 may support sensor 200 operations for meaningful periods of time (e.g., days rather than hours) before needing to be recharged (e.g., via power management 220).

Power management 220 may provide operational power to each of monitors 202-212, microcontroller 216 and memory 218. In one embodiment, battery 222 may be a Li-ion battery rechargeable via universal serial bus (USB) or magnetically. Power management 220 may interface (e.g., via I2C interface 226) to microcontroller 216 so as to provide real-time, state of charge (SoC) remaining within battery 222 along with voltage/current monitoring and alert flags indicating, for example, “charging complete” and “low voltage” warnings.

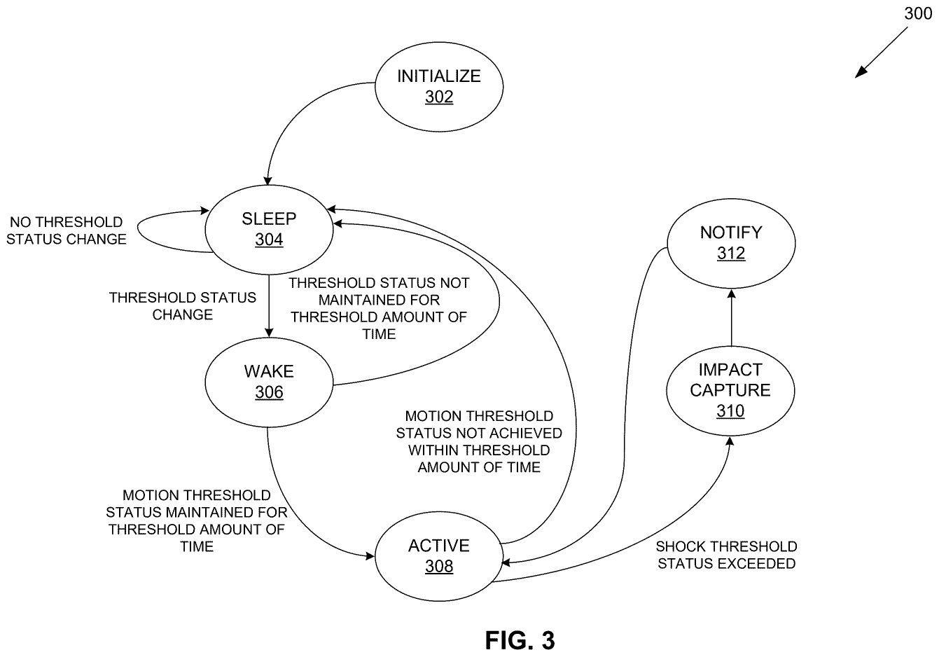

Turning to FIG. 3, state diagram 300 exemplifies the operational states of a sensor (e.g., sensor 200 as discussed above in relation to FIG. 2) of a health monitoring system (e.g., health monitoring system 100 as discussed above in relation to FIG. 1). In state 302, each monitor (e.g., monitors 202-214) of sensor 200 may be initialized for operation (e.g., via configuration code executed within microcontroller 216) along with the initialization/configuration of storage peripherals (e.g., flash memory 218) and wireless networks (e.g., BLE networks 108 and/or 112) each one of which or both may be native to microcontroller 216 thereby obviating the need for external radios. As discussed above, for example, initialization of IMU 202 by microcontroller 216 may comprise the selection of configurable acceleration and duration thresholds required to transition IMU 202 between sleep 304, wake 306, active 308, impact capture 310 and notify 312 modes of operation as shown where each mode of operation may generate varying priority level interrupts to microcontroller 216 that are indicative of acceleration levels detected in each mode of operation.

Once initialized by microcontroller 216 during state 302, sensor 200 may transition to sleep state 304 where power consumption may be minimized while a motion monitor (e.g., IMU 202 of FIG. 2) awaits physical activity resulting in a threshold status change in position, orientation, velocity and/or acceleration. Such a threshold status change (e.g., an acceleration measurement greater than or equal to 0.5 g along any one or more of the x, y or z axes of IMU 202 as determined via a 12.5 Hz sampling rate internal to IMU 202) may be selected by microcontroller 216 during initialization of IMU 202 as the configurable threshold status change event that may trigger a transition from sleep state 304 to wake state 306 as may be indicated by a low-level priority motion interrupt (e.g., as generated by IMU 202 to microcontroller 216 via interface 224 indicative of an acceleration measurement greater than or equal to 0.5 g). If no such threshold status change occurs, then sensor 200 may remain in sleep state 304 to minimize power consumption (e.g., less than 100 μA current draw from battery 222 of power management 220).

Minimization of power consumption during sleep state 304 may, for example, include execution of one or more steps as follows: 1) halting all microcontroller 216 overhead until a motion interrupt from IMU 202 occurs; 2) configuring the sampling rate of IMU 202 to 12.5 Hz; 3) configuring the motion interrupt threshold of IMU 202 to 0.5g along any one or more of the x, y and/or z axes as measured by IMU 202; 3) disabling BLE advertising via wireless interfaces 108 and/or 112; and 4) removing power from memory 218 thereby reducing overall operational power consumption by sensor 200 to approximately zero watts (e.g., between about 0 and 420 μW).

A transition from steep state 304 to wake state 306 may occur when the motion threshold (e.g., 0.5 g along any one or more of the x, y and/or z axes as measured by IMU 202) while in sleep state 304 has been exceeded thereby causing IMU 202 to issue a low-level priority motion interrupt to microcontroller 216. While in wake state 306, microcontroller 216 may transition to its fully operational state whereby BLE advertising via wireless interfaces 108 and/or 112 may be resumed, operational power to motion event storage memory (e.g., flash memory 218) may be restored and IMU 202 may transition to its wake state execution thresholds as may be configured by microcontroller 216 during initialization state 302. In one embodiment, for example, the wake state execution thresholds of IMU 202 may be equivalent to the sleep state execution thresholds of IMU 202 whereby IMU 202 may verify whether or not motion has continued to occur for a threshold amount of time (e.g., 1 second) while in wake state 306. If so, IMU 202 may transition from wake state 306 to active state 308 and may interrupt microcontroller 216 accordingly (e.g., via a low-level priority motion interrupt). If motion has not continued for a threshold amount of time (e.g., 1 second) on the other hand, then IMU 202 may transition from wake state 306 back to sleep state 304.

Minimization of power consumption during wake state 306 may, for example, include execution of one or more steps as follows for a brief duration (e.g., no more than one second): 1) activating all microcontroller 216 activity; 2) maintaining the pre-initialized wake state sampling rate of IMU 202 to 12.5 Hz; 3) maintaining the pre-initialized motion interrupt threshold of IMU 202 to 0.5 g along any one or more of the x, y and/or z axes; and 4) enabling BLE advertising via wireless interfaces 108 and/or 112 thereby reducing overall operational power consumption by sensor 200 to between about 4.2 mW and 8.4 mW (e.g., about 6.3 mW).

A transition from wake state 306 to active state 308 may occur when the motion threshold (e.g., 0.5 g along any one or more of the x, y and/or z axes as measured by IMU 202) while in wake state 306 has been maintained for a threshold amount of time (e.g., 1 second) as may be indicated by a mid-level priority motion interrupt (e.g., as generated by IMU 202 to microcontroller 216 via interface 224). While in active state 308, microcontroller 216 may transition to its fully operational state whereby BLE advertising via wireless interfaces 108 and/or 112 may be resumed, operational power delivered to motion event storage memory (e.g., flash memory 218) may be restored and IMU 202 may transition to its active state execution thresholds as may be initialized by microcontroller 216 during initialization state 302. In one embodiment, the active state execution thresholds of IMU 202 may include threshold parameters (e.g., an acceleration measurement greater than 30 g along any one or more of the x, y or z axes as determined via a 200 Hz sampling rate within IMU 202) that when exceeded within a threshold amount of time (e.g., 30 seconds) may cause IMU 202 to transition from active state 308 to impact capture state 310. In addition, a first-in, first-out (FIFO) buffer (e.g., FIFO 228 of IMU 202) may begin to store the most recent, pre-shock motion event data from IMU 202 in anticipation of a forthcoming shock event as discussed in more detail below. In one embodiment, FIFO 228 may store a configurable number of samples (e.g., 32 samples) representing the most recent, pre-shock motion data (e.g., the 32 samples of motion data immediately preceding a >30 g shock event). If the active state execution thresholds (e.g., an acceleration measurement greater than or equal to 30 g along any one or more of the x, y or z axes as determined via a 200 Hz sampling rate within IMU 202) are not exceeded within a threshold amount of time (e.g., 30 seconds), then IMU 202 may transition from active state 308 back to sleep state 304.

Minimization of power consumption during active state 308 may, for example, include execution of one or more steps as follows for a duration of no more than thirty seconds: 1) activating all microcontroller 216 activity; 2) establishing the pre-initialized active state sampling rate of IMU 202 to 200 Hz; 3) establishing the pre-initialized motion interrupt threshold of IMU 202 to 30 g along any one or more of x, y and/or z axes; and 4) enabling BLE advertising via wireless interfaces 108 and/or 112 thereby reducing overall operational power consumption by sensor 200 to between about 12.6 mW and 16.8 mW (e.g., about 14.7 mW).

A transition from active state 308 to impact capture state 310 may occur when the motion threshold (e.g., 30 g along any one or more of the x, y and/or z axes of IMU 202) while in active state 306 has been exceeded thereby causing an IMU 202 to issue a high-level priority motion interrupt to microcontroller 216. While in impact capture state 310, microcontroller 216 may transition to its fully operational state whereby BLE advertising via wireless interfaces 108 and/or 112 may be resumed, operational power delivered to motion event storage memory (e.g., flash memory 218) may be restored and IMU 202 may transition to its impact capture state execution thresholds as may be initialized by microcontroller 216 during initialization state 302. In one embodiment, the impact capture state execution thresholds of IMU 202 may include threshold parameters (e.g., an acceleration measurement of 30 g along any one or more of the x, y or z axes as determined via a 3200 Hz sampling rate within IMU 202).

In operation during impact capture state 310, the most recent pre-shock memory contents of FIFO buffer 228 within IMU 202 may be transferred to microcontroller 216 as pre-impact waveform data whereby a configurable number of pre-shock motion samples (e.g., 32 samples taken at a 3200 Hz sample rate) that in one embodiment may represent the most recent motion data prior to a >30 g shock event may be stored for processing. An additional configurable number of motion data samples (e.g., 64 data samples taken at a 3200 Hz sampling rate) may be taken by IMU 202 representing a configurable duration (e.g., about 20 ms) of post-shock motion data and then transferred to microcontroller 216 as post-impact waveform data. As per one example, therefore, 32 high-speed samples of motion data immediately preceding a >30 g shock event and the 64 high-speed samples of motion data immediately following the >30 g shock event may be stored as time continuous data (e.g., no time gap between stored pre-impact and post-impact data) and transferred to microcontroller 216 for further processing. Microcontroller 216 may then calculate the maximum acceleration vector magnitude as may be represented within the stored motion data and then may record the impact event within memory (e.g., within flash memory 218). In addition, microcontroller 216 may increment an impact event counter with an updated impact event time stamp and record both in memory (e.g., flash memory 218) as well.

Minimization of power consumption during impact event state 310 may, for example, include execution of one or more steps as follows for a duration between about 30-50 milliseconds: 1) activating all microcontroller activity; 2) establishing the pre-initialized impact capture state sampling rate of IMU 202 to 3200 Hz; 3) establishing the pre-initialized motion interrupt threshold of IMU 202 to 30g along any one or more of the x, y and/or z axes; and 4) enabling BLE advertising via wireless interfaces 108 and/or 112 thereby reducing overall operational power consumption by sensor 200 to between about 21 mW and 33.6 mW (e.g., about 27.3 mW) for a duration between 30-50 ms (e.g., about 40 ms).

In one embodiment, the following structure may represent the impact event data stored within memory (e.g., flash memory 218) by microcontroller for each impact event recorded during impact capture state 310.
// Header (32 bytes)
timestamp; // Relative time in milliseconds
event_number; // Sequential counter (0-65535)
session_id; // Identifies practice/game session
// Impact metrics (16 bytes)
peak_g_x; // Peak acceleration force X-axis (0-200 g)
peak_g_y; // Peak acceleration force Y-axis (0-200 g)
peak_g_z; // Peak acceleration force Z-axis (0-200 g)
peak_g_magnitude; // 3-dimensional magnitude of peak acceleration
duration_ms; // Impact duration in milliseconds
pre_impact_samples; // Number of samples before trigger
post_impact_samples; // Number of samples after trigger
impact_direction; // Encoded direction (frontal/lateral/etc)
reserved1; // Future use
cumulative_g; // Session cumulative G-load
// Waveform data (192 bytes)
samples[32][3]; // 32 samples×3 axes×2 bytes=192 bytes
// Footer (16 bytes)
battery_percent; // Battery level at time of impact
temperature; // Device temperature (optional)
reserved2[10]; // Future use
crc32; // CRC-32 checksum of entire structure

The 192 bytes of impact waveform data above may represent a down-sampled version of the actual number of samples taken to reduce memory storage while simultaneously preserving the impact waveform shape. For example, a down-sampling ratio of 3:1 may reduce the actual sample rate (e.g., 3200 Hz) to an effective sample rate (e.g., 3200/3 or about 1000 Hz) thereby reducing the memory required to store each impact event data structure from 96 samples (576 bytes) to 32 samples (192 bytes). As such, each impact event data structure may be accommodated within a 256-byte storage block of memory.

Once microcontroller 216 completes the impact data capture and post-shock analysis as discussed above, a transition from impact capture state 310 to notify state 312 may be executed whereby microcontroller 216 may first compose an alert packet that may consist of a subset of data (e.g., timestamp, event_number, peak_g_x, peak_g_y, peak_g_z, peak_g_magnitude and duration_ms) that may be derived from the complete impact data structure above. Next, the alert packet may be transmitted by one or more of wireless interfaces 108 and/or 112 (e.g., via a BLE advertisement). In one embodiment, for example, a single alert packet may be transmitted from a slave sensor to a master sensor (e.g., via wireless interface 112 as discussed above in relation to FIG. 1) or a cumulative number of alert packets may be transmitted by a master sensor to a sideline controller (e.g., via wireless interface 108 as discussed above in relation to FIG. 1 above). In an alternate embodiment, for example, each alert packet may be separately transmitted (e.g., via wireless interface 108 or 112) by each sensor directly to a sideline controller (e.g., sideline controller 114 of FIG. 1). As a result, medical staff and coaching personnel may be notified in real time (e.g., via a visual, audible or tactile alert issued by sideline controller 114) so that an evaluation of a potential medical condition (e.g., concussion suffered by an athlete associated with the alarming sensor 200) may be conducted quickly and efficiently thereby obviating the need to wait for hours, days or even weeks for such an evaluation to occur.

Execution state diagram 300 of FIG. 3 exemplifies operation of health monitoring system 100 of FIG. 1 the advantages of which may be summarized and further illuminated as follows. Multiple conditions (e.g., sustained impact events) of an athlete (e.g., athletes 106A and 106B-106n of FIG. 1) may be continuously monitored by hardware (e.g., IMU 202 of FIG. 2) without the intervention of a microcontroller (e.g., microcontroller 216 of FIG. 2) thereby reducing CPU overhead (e.g., the number of operations performed by microcontroller 216 during a specified duration of time) to zero while waiting for impact events and subsequent hardware interrupts to occur. Thus, impact event detection and impact event data capture/reporting may proceed without hindrance that may otherwise be caused by an overloaded CPU handling BLE or flash memory operations simultaneously.

False alarm processing may be executed by IMU 202 during impact capture state 310 after the occurrence of a >30 g shock event through implementation of a duration timer intended to delay the issuance of a shock interrupt to microcontroller 216 until such time that a legitimate shock event may be confirmed by IMU 202. Thus, upon the first occurrence of a >30 g shock event while in active state 308, IMU 202 may start a configurable duration timer (e.g., a 50 ms timer) the expiration of which may cause IMU 202 to re-verify the existence of a >30 g shock event prior to issuing a shock interrupt and prior to impact data capture processing as discussed above. Additionally, a configurable latency timer (e.g., a 40 ms timer) may be initiated subsequent to the first occurrence of a >30 g shock event while in active state 308 to disable further impact data capture processing so as to prevent the occurrence of redundant impact data processing that may be caused by a single shock event. Once the latency timer expires, a configurable observation timer (e.g., a 300 ms timer) may be started such that if another >30 g shock event occurs while the observation timer is running and after the latency timer expires, the shock event may be flagged as a double-impact event.

Impact characterization may also be performed during impact capture state 310. For example, peak acceleration forces may be detected and recorded along each of the x, y and z axes and a 3-dimensional vector magnitude may also be calculated for each impact event. Impact duration and impact direction may also be determined so that impact may be characterized as emanating from the front, side, or back of the athlete or whether the impact was principally vertically oriented thereby enabling rotational vs linear concussion risk assessments. Cumulative load tracking may also be characterized by taking a vector sum of impact forces measured during a typical session (e.g., during a football or soccer game) and issuing an alert when the cumulative load exceeds a safe threshold.

Other aspects and embodiments of the present invention will be apparent to those skilled in the art from consideration of the specification and practice of the invention disclosed herein. It is intended, therefore, that the specification and illustrated embodiments be considered as examples only, with a true scope and spirit of the invention being indicated by the following claims.

What is claimed is:

1. A health monitoring system, comprising:
a plurality of athletes participating in a competitive activity;
a plurality of sensors indirectly coupled to each of the plurality of athletes respectively, wherein each sensor detects a physical aspect of each athlete;
a sideline controller wirelessly coupled to one of the plurality of sensors, wherein the one of the plurality of sensors receives physical aspect data from the remaining sensors and transmits all physical aspect data collected from each of the plurality of athletes to the sideline controller; and
a storage device coupled to the sideline controller and configured to receive the physical aspect data collected from each of the plurality of athletes, wherein the sideline controller may predict potential harm brought to one or more of the plurality of athletes via analysis of the collected physical aspect data.

2. The health monitoring system of claim 1 wherein the physical aspect data is head impact data related to each of the plurality of athletes.

3. The health monitoring system of claim 1 wherein the physical aspect data is hydration data related to each of the plurality of athletes.

4. The health monitoring system of claim 1 wherein the physical aspect data is pulse data related to each of the plurality of athletes.

5. The health monitoring system of claim 1 wherein the physical aspect data is blood pressure data related to each of the plurality of athletes.

6. The health monitoring system of claim 1 wherein the physical aspect data is blood oxygenation data related to each of the plurality of athletes.

7. The health monitoring system of claim 1 wherein the physical aspect data is body temperature data related to each of the plurality of athletes.

8. The health monitoring system of claim 1 wherein the physical aspect data is weight data related to each of the plurality of athletes.

9. The health monitoring system of claim 1 wherein the physical aspect data is body mass index data related to each of the plurality of athletes.

10. The health monitoring system of claim 1 wherein the physical aspect data is foot impact data related to each of the plurality of athletes.

11. A method of monitoring the health of a plurality of athletes, comprising:
initializing a plurality of operational modes associated with an inertial measurement unit (IMU) coupled in proximity to each of the plurality of athletes, each operational mode being indicative of a level of acceleration measured by the IMU;
modifying an overhead of a microcontroller in accordance with each operational mode;
modifying power consumed by the IMU in accordance with each operational mode; and
recording motion detected by the IMU when the measured level of acceleration exceeds a configurable threshold.

12. The method of claim 11, wherein initializing each of the plurality of operational modes includes configuring a sampling rate used by the IMU to be proportional to the magnitude of acceleration as measured by the IMU.

13. The method of claim 12, wherein the power consumed by the IMU is proportional to the magnitude of acceleration measured by the IMU.

14. The method of claim 11, wherein the acceleration measured by the IMU includes acceleration measured about the x, y and z axes of the IMU.

15. The method of claim 14, wherein modifying an overhead of the microcontroller includes,
halting activity of the microcontroller until an interrupt is received from the IMU; and
issuing an interrupt to the microcontroller from the IMU wherein a priority level of the issued interrupt is indicative of the magnitude of acceleration measured by the IMU.

16. The method of claim 15, wherein recording motion includes recording the peak acceleration as measured by the IMU into a data structure after issuance of a high-level priority motion interrupt indicative of a shock event exceeding the configurable threshold.

17. The method of claim 16, wherein recording motion includes recording a duration of the peak acceleration as measured by the IMU after issuance of a high-level priority motion interrupt indicative of a shock event exceeding the configurable threshold.

18. The method of claim 17, wherein recording motion includes recording a number of peak acceleration events as measured by the IMU within a configurable time period after issuance of a high-level priority motion interrupt indicative of a shock event exceeding the configurable threshold.

19. The method of claim 18, wherein recording motion includes buffering pre-impact waveform data immediately prior to a high-level priority motion interrupt indicative of a shock event exceeding the configurable threshold.

20. The method of claim 19, wherein recording motion includes recording post-impact waveform data immediately after a high-level priority motion interrupt indicative of a shock event exceeding the configurable threshold, wherein the post-impact waveform data is time-continuous with the pre-impact waveform data.
